(19) United States
(12) Patent Application Publication
(10) Pub. No.: US 2008/0166593 A1
(43) Pub. Date: Jul. 10, 2008

(54) Organic Electroluminescent Devices

(76) Inventors: Philipp Stoessel, Frankfurt am Main (DE); Holger Heil, Darmstadt (DE); Horst Vestweber, Gilersberg-Winterscheid (DE)

Correspondence Address: CONNOLLY BOVE LODGE & HUTZ, LLP, P O BOX 2207, WILMINGTON, DE, 19899, US

(21) Appl. No.: 11/911,073

(22) PCT Filed: Mar. 20, 2006
(86) PCT No.: PCT/EP06/02532

§ 371 Date: Oct. 9, 2007

(30) Foreign Application Priority Data
Apr. 12, 2005 (EP) 05007958.1

(51) Int. Cl. B32B 9/00; C07C 43/20; C07D 241/38; C07D 487/00; C07D 215/00; C07D 217/00; C07D 213/02; C07D 251/00; C07D 209/04; C07D 233/56; C07D 277/66; C07C 321/30

(52) U.S. Cl. 428/704; 568/632; 544/344; 544/345; 546/139; 546/152; 546/285; 544/215; 548/469; 548/304.4; 548/160; 568/58; 428/411.1

(57) ABSTRACT

The present invention relates to the use of certain organic compounds comprising fused aromatic compounds in organic electronic devices, in particular electroluminescent devices.

In a number of applications of various types which can be ascribed to the electronics industry in the broadest sense, the use of organic semiconductors as functional materials has been reality for some time or is expected in the near future. The general structure of organic electroluminescent devices (OLEDS) is described, for example, in U.S. Pat. No. 4,539,507, U.S. Pat. No. 5,151,629, EP 0676461 and WO 98/27136. However, these devices still always exhibit considerable problems requiring urgent improvement:
1. The operating lifetime is still always short, in particular in the case of blue emission, meaning that it has hitherto only been possible to achieve simple applications commercially.
2. In some cases, use is made of mixtures of isomeric compounds, which may have different physical properties (glass transition temperature, glass formation properties, absorption, photoluminescence).

Since these stereoisomers in some cases also have different vapour pressures at the processing temperature, uniform, reproducible production of the organic electronic device is not possible. This problem is described in detail, for example, in unpublished application EP 04026402.0.
3. The compounds used are in some cases only sparingly soluble in common organic solvents, which makes their purification during synthesis more difficult, but also makes cleaning of the plants in the case of the production of the organic electronic devices more difficult.
4. In many host materials in accordance with the prior art which consist of pure hydrocarbons, the hole mobility and the stability to holes are not sufficiently high and must be improved.

The closest prior art can be regarded as the use of various fused aromatic compounds, in particular anthracene or pyrene derivatives, as host materials, in particular for blue-emitting electroluminescent devices. The host material disclosed in the prior art is 9,10-bis(2-naphthyi)anthracene (U.S. Pat. No. 5,935,721). Further anthracene derivatives which are suitable as host materials are described, for example, in WO 01/076323, WO 01/021729, WO 04/013073, WO 04/018588, WO 03/087023 or WO 04/018587. Host materials based on aryl-substituted pyrenes and chrysenes are described in WO 04/016575, which in principle also encompasses corresponding anthracene and phenanthrene derivatives. WO 03/095445 and CN 1362464 describe 9,10-bis(1-naphthyl)anthracene derivatives for use in OLEDs. For high-quality applications, it is necessary to have improved host materials available. The above-mentioned compounds are particularly problematical if they form atropisomers and thus lead to poorly reproducible results during device production.

The above-mentioned prior art confirms that the host material plays a crucial role in the function of organic electroluminescent devices. Thus, there continues to be a demand for improved materials, in particular host materials for blue-emitting OLEDs, which lead to good efficiencies and at the same time to long lifetimes in organic electronic devices and lead to reproducible results in the production and operation of the devices. Surprisingly, it has been found that organic electroluminescent devices which contain compounds of fused aromatic rings which have been functionalised by aryloxy or thloaryloxy substituents have significant improvements over the prior art. These materials enable an increase in the efficiency and service life of the organic electronic device compared with materials in accordance with the prior art. Since these materials cannot exhibit atropisomerism about the aryl-O-aryl bonds leading to diastereomers, reproducible production of the organic electronic devices continues to be possible. The present invention therefore relates to the use of these materials in organic electronic devices.

JP 2000/021571 describes the use of 9,10-bis(aryloxy)- and 9,10-bis(aryl-thio)anthracenes in OLEDs. A particular advantage of these compounds is not evident.

JP 11111458 describes dianthracene derivatives which may also be substituted, inter alia, by aryloxy substituents. The effect of these compounds is attributed to the two anthracene units linked to one another. A particular advantage of the aryloxy-substituted compounds over the numerous other compounds mentioned is not evident, so it must be assumed that this substituent is only mentioned here by chance alongside a large number of other possible substituents.

US 2004/185298 describes 1,9-peri-(9′-anthrylene)-10-(9′-anthryl)anthracene derivatives which may in principle also carry aryloxy substituents in addition to numerous other substituents. However, aryloxy-substituted compounds are not listed, and consequently it is not possible to deduce an advantage of such compounds. In particular, the compounds described are used as dopants for red-emitting OLEDs. The extended fused ring system means that they are not suitable for blue-emitting OLEDs.

WO 01121729 describes OLEDs which comprise both styrylamines and also certain anthracene derivatives in one layer. Two anthracene units here are bridged via various bridges, inter alia also oxygen or sulfur. In the subsequent application WO 01/76323, an electron-transport compound is also used in the same layer in addition to the above-mentioned compounds. This suggests that the use of a separate electron-transport compound is necessary for good results, which significantly restricts the utility of these anthracene derivatives.

US 6582837 describes in general 9-(1-naphthyl)anthracene derivatives for use in OLEDs, in particular those which are substituted by diarylamino groups. These compounds may carry further substituents, inter alia also aryloxy, in positions 1-8 and 10 on the anthracene. However, aryloxy-substituted compounds are not disclosed explicitly, and consequently an advantage of such compounds cannot be deduced. This suggests that the aryloxy substituent is only mentioned here by change alongside a large number of other possible substituents.

JP 20051008600 describes anthracene derivatives which are substituted by tetrahydronaphthalene in the 9,10-position. The anthracene units here may carry further substituents, inter alia also phenoxy or naphthoxy groups, in the 2- or 2,6-position. However, a particular advantage of the phenoxy- or naphthoxy-substituted compounds over differently substituted compounds is not evident, and the particular effect of these compounds is not based on the phenoxy or naphthoxy groups, but instead on the tetrahydronaphthalene units.

WO 04/018587, WO 04/013073, JP 2003/313156, WO 02/43448 and WO 01/72673 also describe anthracene derivatives which, besides numerous other substituents, may also carry aryloxy substituents in the 1-8 position on the anthracene. However, no such structures are given, and no advantage of aryloxy substitution over other substitutions is evident, and it can consequently be assumed that these substituents are only disclosed by chance in a list alongside numerous other substituents.

The invention relates to organic electroluminescent devices comprising cathode, anode and at least one organic layer which comprises at least one compound of the formula (1)

Ar¹—X—Ar²—Ar³ Formula (1)

where the following applies to the symbols used:
Ar¹, Ar³ are on each occurrence, identically or differently, an aromatic or heteroaromatic ring system, which may be substituted by one or more radicals R;
Ar² is on each occurrence, identically or differently, a fused aryl or heteroaryl group having at least 14 aromatic ring atoms, which may be substituted by one or more radicals R;
X is on each occurrence O, S, Se or Te;
R is on each occurrence, identically or differently, H, F, Cl, Br, I, a straight-chain alkyl, alkoxy or thioalkoxy chain having 1 to 40 C atoms or a branched or cyclic alkyl, alkoxy or thioalkoxy chain having 3 to 40 C atoms, each of which may be substituted by R¹, and in which one or more non-adjacent C atoms may be replaced by N-R¹, O, S, O—CO—O, CO—O, Si(R¹)₂, CO, CO—N(R¹)₂, —CR¹═CR¹— or —C—C— and in which, in addition, one or more H atoms may be replaced by F, Cl, Br, I or CN, or an aromatic or heteroaromatic ring system, which may also be substituted by one or more radicals R¹, or a combination of two, three or four of these systems; two or more radicals R here may with one another also form a further mono- or polycyclic, aliphatic or aromatic ring system;
R¹ is on each occurrence, identically or differently, H or an aliphatic or aromatic hydrocarbon radical having 1 to 20 C atoms;
with the proviso that at most one of the groups Ar¹, Ar² and Ar³ represents an anthracene, and furthermore with the proviso that if Ar² represents an anthracene, the group Ar¹-X is not bonded in the 2-position.

Although this is evident from the above description, it is again emphasised here that a plurality of radicals R may with one another also form a ring system, also for example and in particular between the groups Ar¹ and Ar³.

The compound of the formula (1) preferably has a glass transition temperature T_{g} of greater than 70° C., particularly preferably greater than 100° C., very particularly preferably greater than 130° C.

For the purposes of this invention, an aromatic ring system contains 6 to 40 C atoms in the ring system. For the purposes of this invention, a heteroaromatic ring system contains 2 to 40 C atoms and at least one heteroatom in the ring system, with the proviso that the total number of C atoms and heteroatoms is at least 5. The heteroatoms are preferably selected from N, O and/or S. For the purposes of this invention, an aromatic or heteroaromatic ring system is taken to mean a system which does not necessarily contain only aryl or heteroaryl groups, but in which a plurality of aryl or heteroaryl groups may also be interrupted by a short, non-aromatic unit (less than 10% of the atoms other than H, preferably less than 5% of the atoms other than H), such as, for example, an sp³-hybridised C, N or O atom. Thus, for example, aromatic ring systems for the purposes of this invention are also taken to mean systems such as 9,9′-spirobifluorene, 9,9-diarylfluorene, triarylamine, diaryl ether, etc. The aromatic or heteroaromatic ring system or a part thereof may also be a fused group here in the sense of the following definition.

For the purposes of this invention, a fused aryl or heteroaryl group is taken to mean a ring system having 10 to 40 aromatic ring atoms in which at least two aromatic or heteroaromatic rings are fused to one another, i.e. have at least one common edge and a common aromatic n-electron system. These ring systems may be substituted by R or unsubstituted. Examples of fused aromatic or heteroaromatic ring systems are naphthalene, quinoline, isoquinoline, anthracene, phenanthrene, pyrene, perylene, chrysene, acridine, etc., while biphenyl, for example, is not a fused aryl group since there is no common edge between the two ring systems therein. Fluorene, for example, is likewise not a fused aromatic ring system since the two phenyl units therein do not form a common aromatic electron system.

For the purposes of the present invention, a C₁- to C₄₀-alkyl group, in which individual H atoms or CH₂ groups may also be substituted by the above-mentioned groups, is particularly preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. A C₁- to C₄₀-alkoxy group is particularly preferably taken to mean methoxy, ethoxy, n-propoxy, i-propoxy, n-butoxy, i-butoxy, s-butoxy, t-butoxy or 2-methyl-butoxy. An aromatic or heteroaromatic ring system having 5-40 aromatic ring atoms, which may also in each case be substituted by the above-mentioned radicals R and which may be linked to the aromatic or heteroaromatic ring via any desired positions, is in particular taken to mean groups derived from benzene, naphthalene, anthracene, phenanthrene, pyrene, chrysene, perylene, fluoranthene, naphthacene, pentacene, benzopyrene, biphenyl, biphenylene, terphenyl, terphenylene, fluorene, spirobifluorene, diphenyl ether, triphenylamine, dihydrophenanthrene, dihydropyrene, tetrahydropyrene, cis- or trans-indenofluorene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-iazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The fused aryl or heteroaryl group Ar² preferably contains three, four, five or six aromatic or heteroaromatic units, which are in each case fused to one another via one or more common edges and thus form a common aromatic system and which may be substituted by R or unsubstituted. The fused aryl or heteroaryl group Ar² particularly preferably contains three, four or five aromatic or heteroaromatic units, in particular three or four aromatic or heteroaromatic units, which are in each case fused to one another via one or more common edges and thus form a common aromatic system and which may be substituted by R or unsubstituted. The aromatic and heteroaromatic units fused to one another are particularly preferably selected from benzene, pyridine, pyrimidine, pyrazine and pyridazine, each of which may be substituted by R or unsubstituted, very particularly preferably benzene and pyridine, especially benzene.

The fused aryl or heteroaryl groups Ar² are particularly preferably selected from the group consisting of anthracene, acridine, phenanthrene, phenanthroline, pyrene, naphthacene, chrysene, pentacene and perylene, each of which may optionally be substituted by R. Substitution by R may be appropriate in order to obtain more highly soluble compounds. The fused aromatic ring systems are particularly preferably selected from the group consisting of anthracene, phenanthrene, pyrene, naphthacene and perylene, in particular anthracene, phenanthrene, pyrene and perylene, each of which may optionally be substituted by R. The units Ar¹ and Ar³ are preferably linked to anthracene via the 1,10-position, the 9,10-position or via the 1,4-position, particularly preferably via the 9,10-position. The linking to pyrene preferably takes place via the 1,6-, 1,8-, 1,3- or 2,7-position, particularly preferably via the 1,6- or via the 2,7-position. The linking to phenanthrene preferably takes place via the 2,7-, 3,6-, 9,10-, 2,9- or 2,10-position, particularly preferably via the 2,7- or via the 3,6-position. The linking to perylene preferably takes place via the 3,4-, 3,9- or 3,10-position, particularly preferably via the 3,9- or via the 3,10-position.

Preferred compounds of the formula (1) are thus the following compounds of the formulae (2) to (14), each of which may also be substituted by R, and where the symbols used have the same meaning as described above:

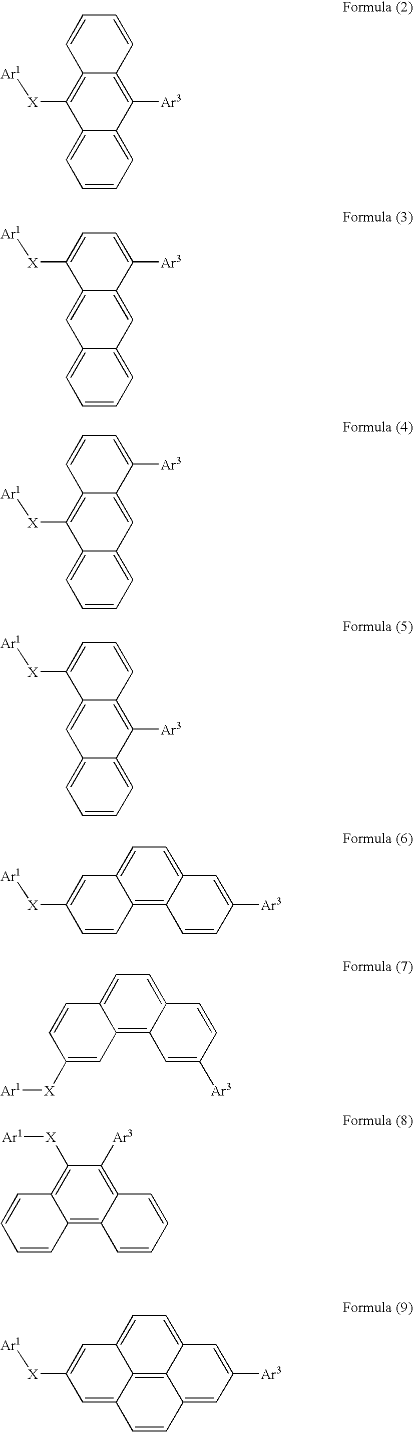

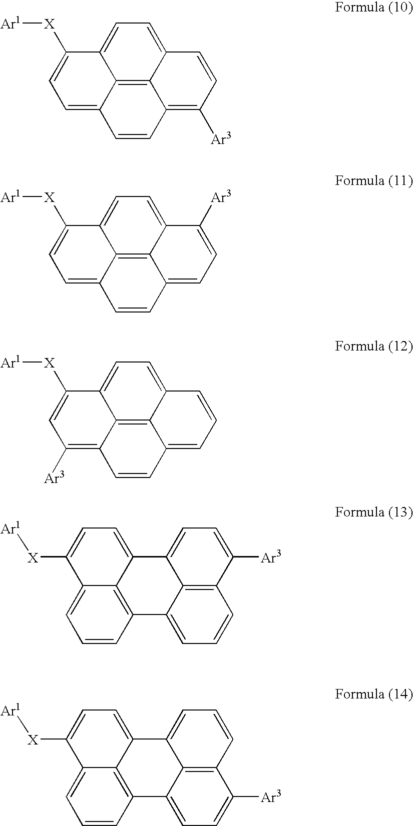

Of these, very particular preference is given to the compounds of the formulae (2), (6), (7), (8), (9), (10) and (11).

It is also possible and admissible here for more than one group Ar¹-X, for example two, three or four groups Ar¹-X, to be bonded to the fused aromatic unit. It is likewise possible and admissible for more than one group Ar³, for example two, three or four groups Ar³, to be bonded to the fused aromatic unit.

The anthracene and pyrene units here are preferably unsubstituted, apart from the groups X and Ar³. The phenanthrene units are likewise preferably unsubstituted, apart from the groups X and Ar³, or compounds of the formulae (6) and (7) also carry substituents in position 9 and/or 10, or compounds of the formula (8) also carry substituents in position 2 and/or 7 or in position 3 and/or 6. The perylene units are likewise preferably unsubstituted, apart from the groups X and Ar³, or compounds of the formula (13) also carry substituents in position 4 and/or 9, or compounds of the formula (14) also carry substituents in position 4 and/or 10.

Preferred groups Ar¹ and Ar³ are, identically or differently on each occurrence, simple or fused aryl or heteroaryl groups having 5 to 16 aromatic ring atoms or spirobifluorene. Particularly preferred groups Ar¹ and Ar³ are simple or fused aryl or heteroaryl groups having 6 to 14 aromatic ring atoms. These may each be substituted by R or unsubstituted. Ar¹ and Ar³ are especially preferably simple or fused aryl groups. Very particularly preferably, at least one of the two groups Ar¹ and Ar³ is a fused aryl or heteroaryl group, in particular the group Ar³. Very particularly preferably, both groups Ar¹ and Ar³ are fused aryl or heteroaryl groups, in particular fused aryl groups.

Preferred groups X are O, S or Se, particularly preferably O or S, very particularly preferably O.

Preferred radicals R are, if present, identically or differently on each occurrence, H, F, a straight-chain alkyl or alkoxy chain having 1 to 10 C atoms or a branched alkyl or alkoxy chain having 3 to 10 C atoms, each of which may be substituted by R¹ and in which one or more non-adjacent C atoms may be replaced by N-R¹, O, S, —CR¹-CR¹— or —C═C—, and in which, in addition, one or more H atoms may be replaced by F or CN, or an aromatic or heteroaromatic ring system having 5 to 16 aromatic ring atoms, which may also be substituted by one or more radicals R¹, or a combination of two or three of these systems; two or more radicals R here may with one another also form a further mono- or polycyclic, aliphatic or aromatic ring system. Particularly preferred radicals R are, if present, identically or differently on each occurrence, H, F, a straight-chain alkyl chain having 1 to 5 C atoms or a branched alkyl chain having 3 to 5 C atoms and in which one or more non-adjacent C atoms may be replaced by —CR1═CR¹— or —C═C—, and in which, in addition, one or more H atoms may be replaced by F, or an aryl or heteroaryl group having 5 to 10 aromatic ring atoms, which may also be substituted by one or more radicals R¹, or a combination of two of these systems; two or more radicals R here may with one another also form a further mono- or polycyclic, aliphatic or aromatic ring system.

Examples of suitable compounds of the formula (1) are the structures (1) to (86) shown below.

```

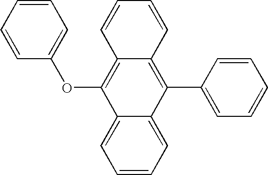

  (1)

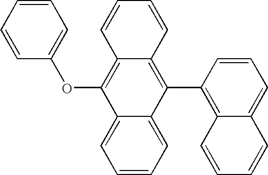

  (2)

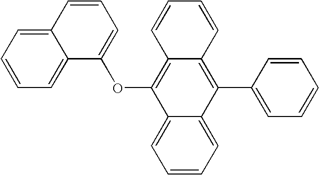

  (3)

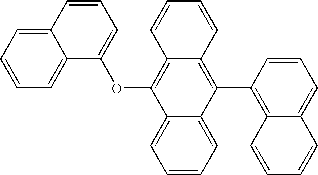

  (4)

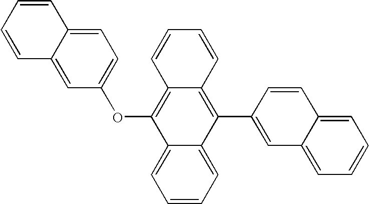

  (5)

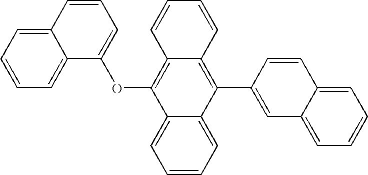

  (6)

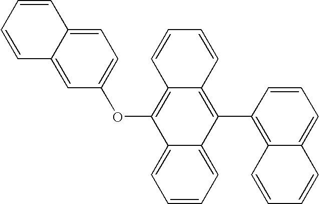

  (7)

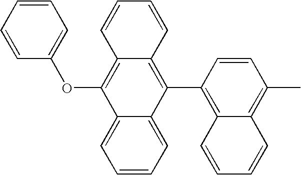

  (8)

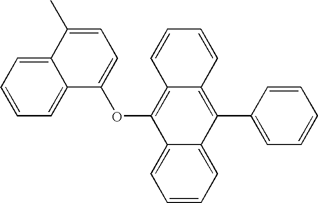

  (9)

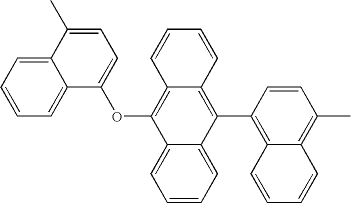

(10)

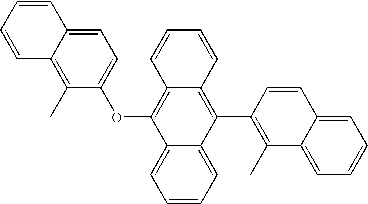

(11)

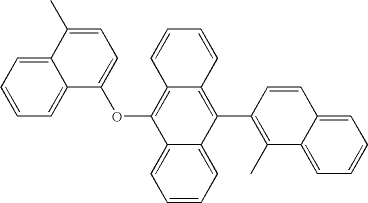

(12)

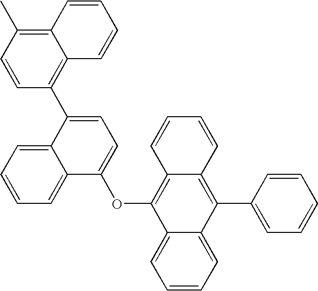

(13)

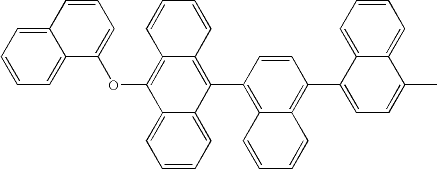

(14)

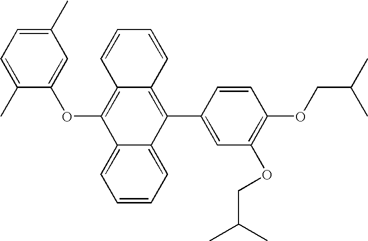

(15)

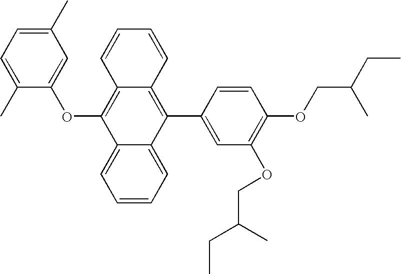

(16)

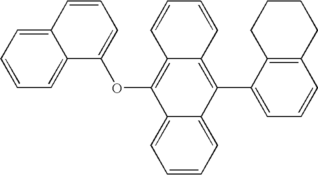

(17)

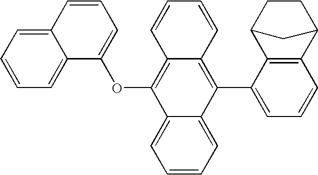

(18)

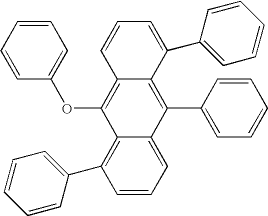

(19)

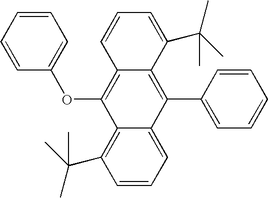

(20)

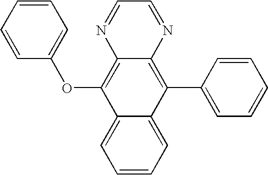

(21)

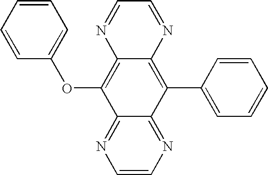

(22)

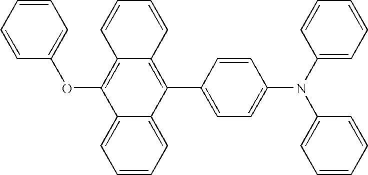

(23)

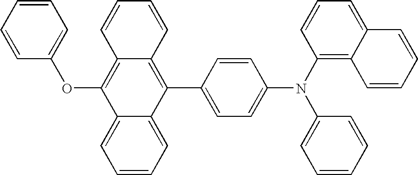

(24)

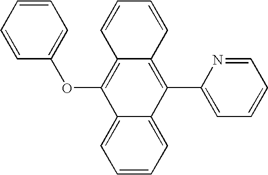

(25)

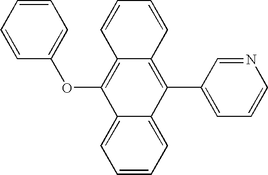

(26)

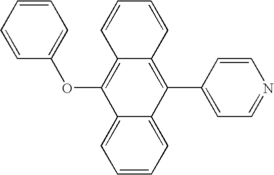

(27)

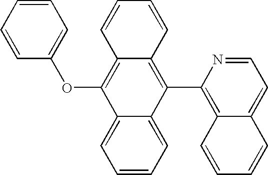

(28)

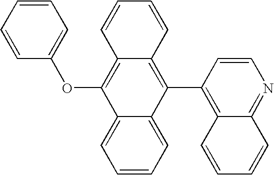

(29)

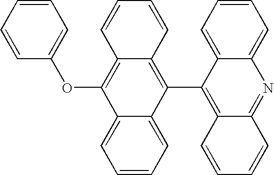

(30)

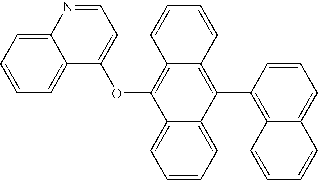

(31)

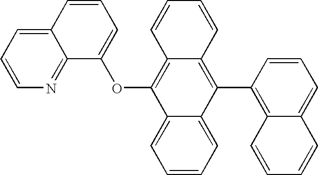

(32)

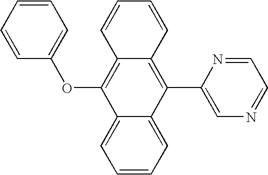

(33)

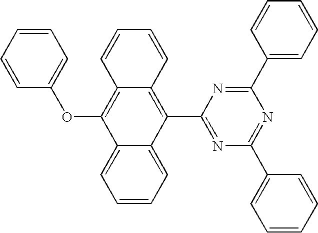

(34)

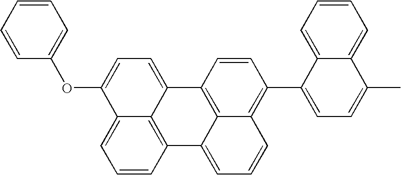

(35)

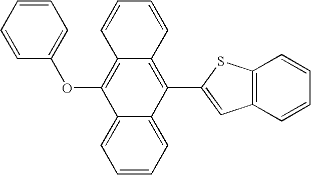

(36)

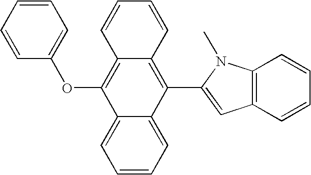

(37)

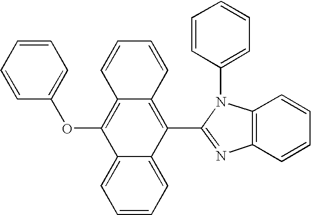

(38)

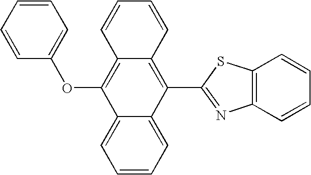

(39)

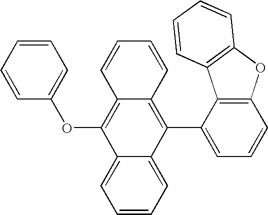

(40)

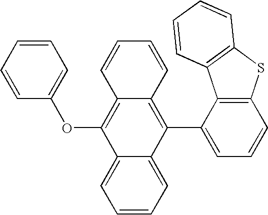

(41)

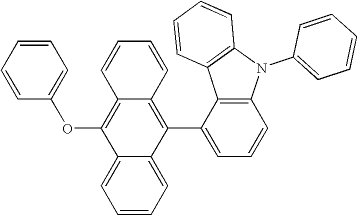

(42)

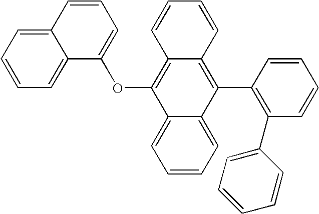

(43)

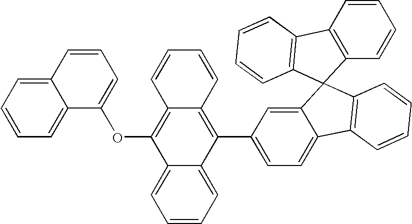

(44)

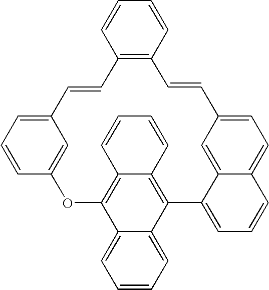

(45)

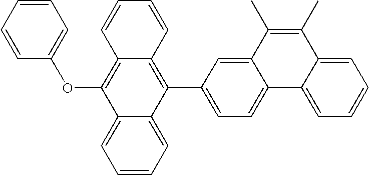

(46)

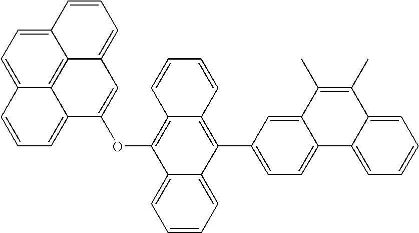

(47)

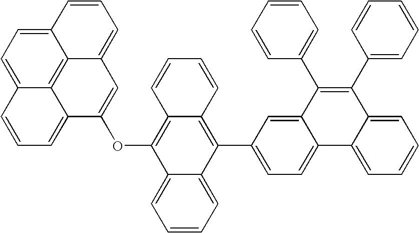

(48)

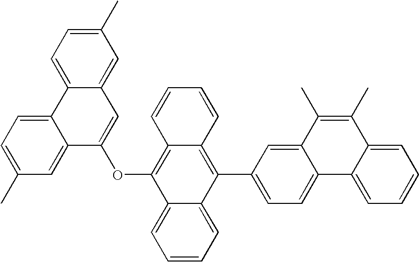

(49)

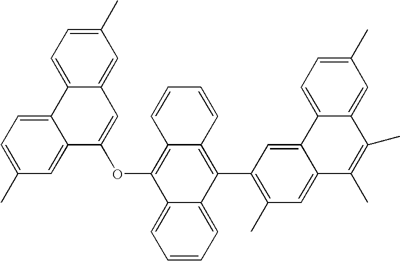

(50)

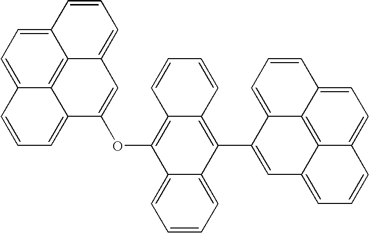

(51)

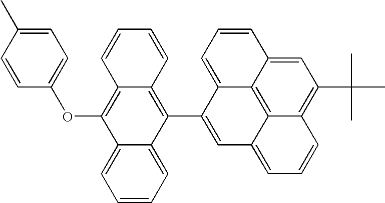

(52)

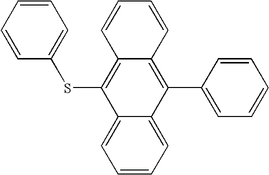

(53)

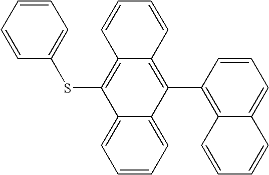

(54)

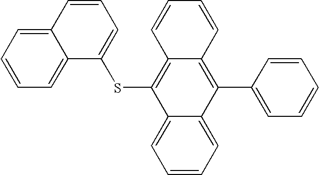

(55)

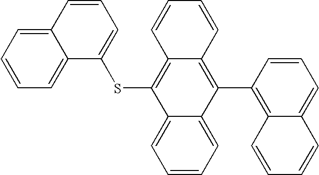

(56)

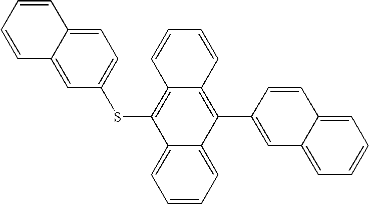

(57)

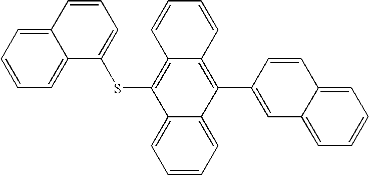

(58)

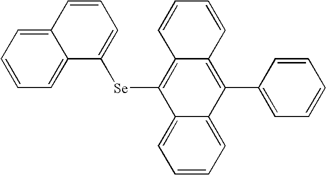

(59)

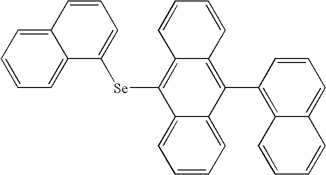

(60)

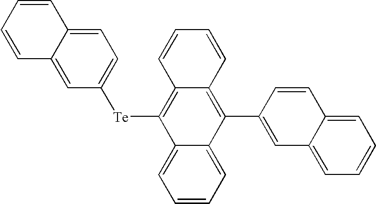

(61)

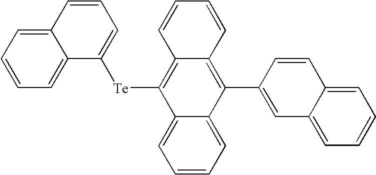

(62)

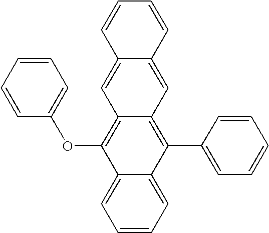

(63)

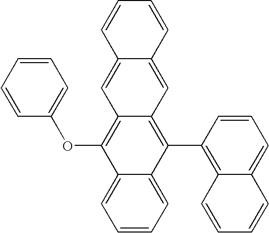

(64)

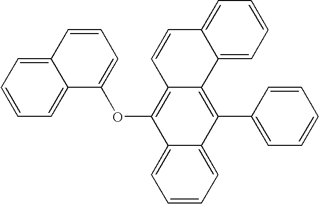

(65)

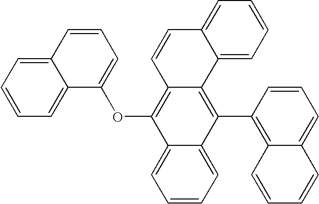

(66)

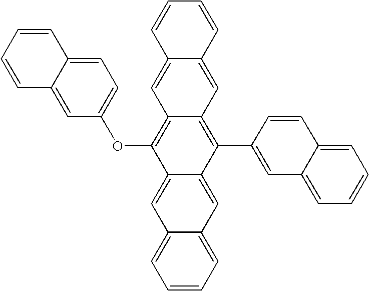

(67)

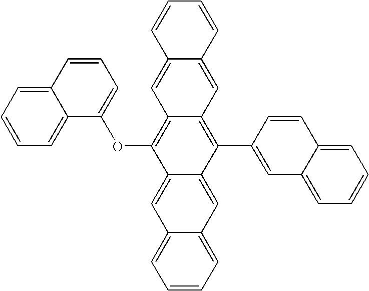

(68)

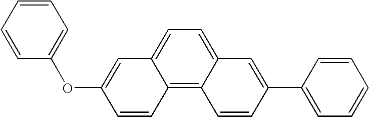

(69)

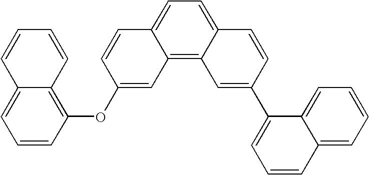

(70)

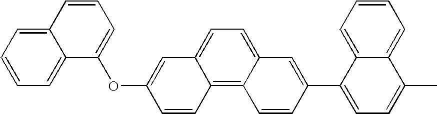

(71)

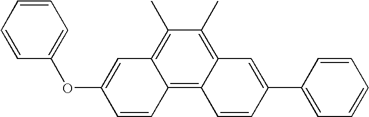

(72)

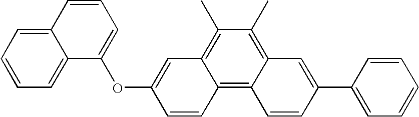

(73)

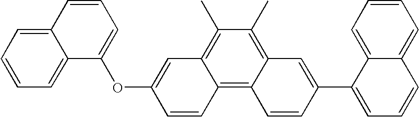

(74)

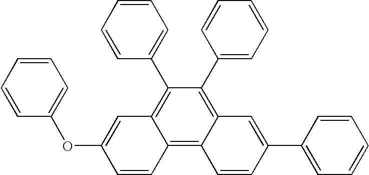

(75)

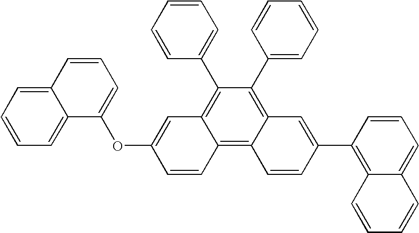

(76)

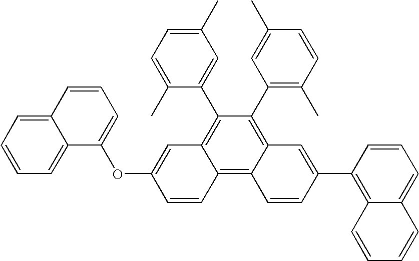

(77)

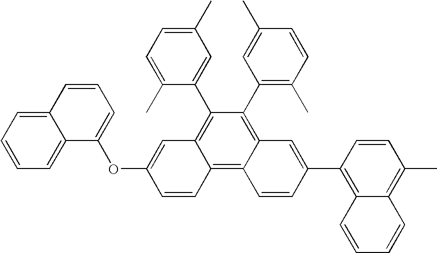

(78)

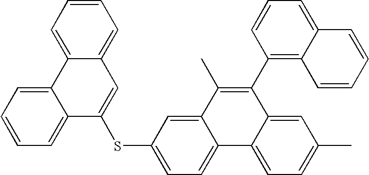

(79)

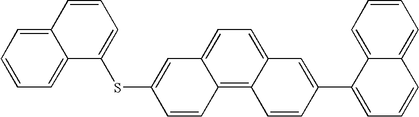

(80)

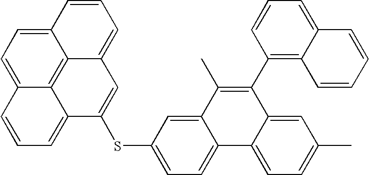

(81)

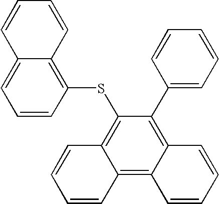

(82)

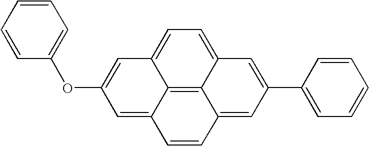

(83)

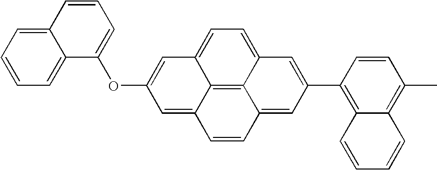

(84)

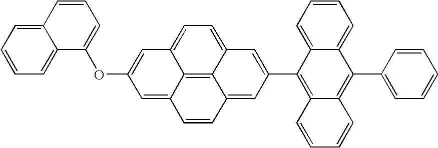

(85)

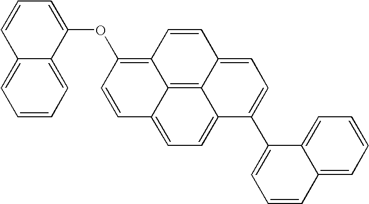

(86)
```

The compounds of the formula (1) can be synthesised by standard methods of organic chemistry. Thus, for example, it is possible firstly to construct the unit Ar²-Ar³ by coupling a halogen compound of the first aromatic unit to a boronic acid derivative of the other aromatic unit in a Suzuki coupling with palladium catalysis. It is also possible to use a tin derivative in a Stille coupling with palladium catalysis or further transition metal-catalysed coupling reactions. In a next step, the aromatic unit Ar² can be halogenated, for example by bromination using NBS or using bromine. Selective halogenation is possible if the aromatic unit Ar³ is appropriately substituted so that no halogenation can take place here. The aryloxy substituent (or corresponding S, Se or Te substituents) can be introduced by reaction of this compound with a phenol or corresponding sulfur, selenium or tellurium compounds. This reaction can be carried out, for example, as an aromatic nucleophilic substitution or with copper catalysis under the conditions of the Ullmann coupling (F. Ullmann et al., Chem. Ber. 1905, 38, 2211-2212) or with palladium catalysis under the conditions of the Hartwig-Buchwald coupling (G. Mann et al., J. Am. Chem. Soc. 1999, 121, 3224-3225; A. Aranyos et al., J. Am. Chem. Soc, 1999, 121, 4369-4378). A further possibility is conversion of the halogenated aromatic unit into the corresponding Grignard or aryllithium reagent and further reaction thereof with diaryidithiols, -diselenides or -ditellurides.

Compounds of the formula (1a) in which at least one of the groups Ar¹ and Ar³ contains a fused aryl or heteroaryl group or spirobifluorene are novel and are therefore likewise a subject-matter of the present invention.

The invention relates to compounds of the formula (1a)

Ar¹—X—Ar²—Ar³ Formula (1a)

where the following applies for the symbols used:
Ar¹, Ar³ are on each occurrence, identically or differently, an aromatic or heteroaromatic ring system, which may be substituted by one or more radicals R, where at least one of the two groups Ar¹ and/or Ar³ contains a fused aryl or heteroaryl group or a spirobifluorene;

the other symbols are as defined above; with the proviso that at most one of the groups Ar¹, Ar² and Ar³ represents an anthracene, and furthermore with the proviso that if Ar² represents an anthracene, the group Ar¹-X is not bonded in the 2-position.

The same preferences as described above apply for the groups Ar² and X. Preferred compounds of the formula (1a) are thus compounds of the formulae (2) to (12) in which at least one of the groups Ar¹ and/or Ar³ contains at least one fused aryl or heteroaryl group or a spirobifluorene, each of which may be substituted by R.

In a preferred embodiment of the invention, the fused aryl or heteroaryl group Ar¹ or Ar³ is selected from the group consisting of naphthalene, quinoline, isoquinoline, quinoxaline, anthracene, acridine, phenanthrene, phenanthroline, pyrene, chrysene, naphthacene, pentacene and perylene, with the proviso that at most one of the groups Ar¹, Ar² and Ar³ represents anthracene. Particularly preferred fused aryl or heteroaryl groups Ar¹ and/or Ar³ are selected from the group consisting of naphthalene, quinoline, isoquinoline, anthracene, phenanthrene, pyrene and perylene, very particularly preferably naphthalene and phenanthrene.

In a preferred embodiment of the invention, the group Ar³ contains at least one fused aryl or heteroaryl group, which may be substituted by R. In a particularly preferred embodiment of the invention, both groups Ar¹ and Ar³ contain at least one fused aryl or heteroaryl group, each of which may be substituted by R.

The invention furthermore relates to the use of compounds of the formula (1a) in organic electronic devices, in particular in organic electroluminescent devices.

The organic electroluminescent device comprises, as described above, anode, cathode and at least one organic layer comprising at least one compound of the formula (1). At least one of the organic layers here is an emission layer. It may also be preferred for the organic electronic device to comprise further layers. Apart from the emission layer, these may be, for example: hole-injection layer, hole-transport layer, electron-transport layer and/or electron-injection layer. However, it should be pointed out at this point that each of these layers does not necessarily have to be present.

Thus, in particular on use of compounds of the formula (1) in the emission layer, very good results are furthermore obtained if the organic electroluminescent device does not comprise a separate electron-transport layer and the emitting layer is directly adjacent to the electron-injection layer or to the cathode. It may likewise be preferred for the organic electroluminescent device not to comprise a separate hole-transport layer and for the emitting layer to be directly adjacent to the hole-injection layer or to the anode.

Preferred materials for an optionally present electron-transport layer are metal complexes containing aluminium or gallium, polypodal metal complexes (for example in accordance with WO 04/081017), ketones, phosphine oxides or sulfoxides (for example in accordance with WO 051084081 and WO 05/084082). Particular preference is given to ketones and phosphine oxides.

The compound of the formula (1) is particularly preferably employed in the emission layer. It can be employed as pure substance, but is preferably employed in combination with a dopant. The dopant is preferably selected from the class of the monostyrylamines, distyrylamines, tristyrylamines, tetrastyrylamines and arylamines. A monostyrylamine is taken to mean a compound which contains a styryl group and at least one, preferably aromatic, amine. A distyrylamine is taken to mean a compound which contains two styryl groups and at least one, preferably aromatic, amine. A tristyrylamine is taken to mean a compound which contains three styryl groups and at least one, preferably aromatic, amine. A tetrastyrylamine is taken to mean a compound which contains four styryl groups and at least one, preferably aromatic, amine. For the purposes of this invention, an arylamine or an aromatic amine is taken to mean a compound which contains three aromatic or heteroaromatic ring systems bonded directly to the nitrogen. The styryl groups are particularly preferably stilbenes, which may also be further substituted. Particularly preferred dopants are selected from the class of the tristyrylamines. Examples of dopants of this type are substituted or unsubstituted tristilbenamines or the dopants described in WO 06/000388, WO 06/000389, WO 06/000390 and unpublished patent application EP 04028407.7.

The proportion of the compound of the formula (1) in the mixture is usually between 1 and 99.9% by weight, preferably between 50 and 99.5% by weight, particularly preferably between 80 and 99% by weight, in particular between 90 and 99% by weight. The proportion of the dopant is correspondingly between 0.1 and 99% by weight, preferably between 0.5 and 50% by weight, particularly preferably between 1 and 20% by weight, in particular between 1 and 10% by weight.

Preference is furthermore given to organic electroluminescent devices which are characterised in that a plurality of emitting compounds are used in the same layer or a plurality of emitting layers are present, where at least one of the layers comprises at least one compound of the formula (1). This device particularly preferably has overall a plurality of emission maxima between 380 nm and 750 nm, so that overall white emission results. Emitting compounds which can be employed here are both those which exhibit fluorescence and those which exhibit phosphorescence.

The compounds of the formula (1) are furthermore suitable for use as electron-transport material, in particular in an electron-transport layer, in fluorescent and phosphorescent electroluminescent devices. They are likewise suitable for use as hole-blocking material, in particular in a hole-blocking layer, in fluorescent and phosphorescent electroluminescent devices.

The compounds of the formula (1) are furthermore suitable for use as hole-transport material, in particular in a hole-transport layer, in fluorescent and phosphorescent electroluminescent devices. This applies, in particular, if more than one group Ar¹-X is present in the molecule.

Preference is furthermore given to an organic electronic device, characterised in that one or more layers are coated by a sublimation process. The materials here are applied by vapour deposition in vacuum sublimation units at a pressure below 10⁻⁵ mbar, preferably below 10⁻⁶ mbar, particularly preferably below 10⁻⁷ mbar.

Preference is likewise given to an organic electronic device, characterised in that one or more layers are coated by the OVPD (organic vapour phase deposition) process or with the aid of carrier-gas sublimation. The materials here are generally applied at a pressure between 10⁻⁵ mbar and 1 bar.

Preference is furthermore given to an organic electronic device, characterised in that one or more layers are produced from solution, such as, for example, by spin coating, or using any desired printing process, such as, for example, screen printing, flexographic printing or offset printing, but particularly preferably by LITI (light induced thermal imaging, thermal transfer printing) or ink-jet printing.

The emitting devices described above have the following surprising advantages over the prior art:
1. The stability of corresponding devices is higher compared with systems in accordance with the prior art, which is particularly evident in a longer lifetime of the OLED. This effect may be due to higher stability to holes of the compounds of the formula (1) compared with compounds in accordance with the prior art.
2. In contrast to compounds used to date, some of which were difficult to purify owing to their poor solubility, the compounds of the formula (1) are readily soluble and therefore easier to purify and also easier to process from solution.
3. Materials in accordance with the prior art in some cases form atropisomers, which result in problems with reproducibility, as already explained above. The introduction of the group X makes the formation of diastereomeric atropisomers about the aryl-X-aryl bond impossible, thus enabling the reproducible production of the device. In the present application text and also in the examples following below, the aim is the use of the compounds of the formula (1) in relation to OLEDs and the corresponding displays. In spite of this restriction of the description, it is possible for the person skilled in the art, without a further inventive step, to use the compounds of the formula (1) also for further uses in other electronic devices, for example for organic field-effect transistors (O-FETs), organic thin-film transistors (O-TFTs), organic light-emitting transistors (O-LETs), organic integrated circuits (O-ICs), organic solar cells (O-SCs), organic field-quench devices or organic laser diodes (O-lasers), to mention but a few applications. The present invention likewise relates to the use of the compounds according to the invention in the corresponding devices and to these devices themselves.

The invention is explained in greater detail by the following examples, without wishing to be restricted thereby.

### EXAMPLES

The following syntheses are, unless indicated otherwise, carried out under a protective-gas atmosphere. The starting materials can be purchased from ALDRICH (4-methyinaphthalene-1-boronic acid, 9-bromoanthracene, phenol, 4-phenylphenol, 2-phenylphenol, palladium(II) acetate, tri-o-tolyl-phosphine, inorganics, solvents).

### Example 1

### 10-(4-Methyinaphth-1-yl)-9-(phenoxy)anthracene (H1)

a) Synthesis of 9-(4-methyinaphth-1-yl)anthracene

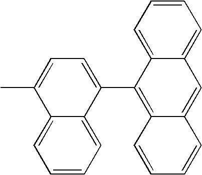

3.6 g (11.7 mmol) of tri-o-tolylphosphine and then 437 mg (1.9 mmol) of palladium(II) acetate are added to a suspension of 93.0 g (500 mmol) of 4-methylnaphthalene-1-boronic acid, 100.0 g (389 mmol) of 9-bromoanthracene and 212.3 g (1 mol) of tripotassium phosphate in a mixture of 400 ml of dioxane, 600 ml of toluene and 1000 ml of water with good stirring, and the mixture is heated under reflux for 16 h. After the reaction mixture has been cooled, the organic phase is separated off and washed three times with 500 ml of water. The organic phase is subsequently filtered through silica gel and evaporated to dryness. The oil which remains is taken up in 1000 ml of ethanol and brought into solution under reflux. After cooling, the colourless solid is filtered off with suction, recrystallised again from 1000 ml of ethanol and finally dried under reduced pressure. Yield: 103.0 g (83.1% of theory), about 96% according to ¹H-NMR.

b) Synthesis of 9-bromo-10-(4-methyinaphth-1-yl)anthracene

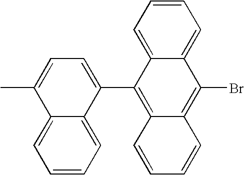

A mixture of 18.0 ml (352 mmol) of bromine in 100 ml of dichloromethane is added dropwise with good stirring at −5° C. to a solution of 102.0 g (320 mmol) of 9-(4-methyinaphth-1-yl)anthracene in 2000 ml of dichloromethane, and the mixture is stirred at room temperature for 12 h. The suspension is subsequently diluted with 1000 ml of ethanol, and a solution of 15 g of sodium sulfite in 500 ml of water is added. The precipitated solid is filtered off with suction and washed with 500 ml of a mixture of water and ethanol (1:1, v:v) and then three times with 200 ml of ethanol. After washing twice with 1000 ml of boiling ethanol each time, the solid is dried under reduced pressure. Yield: 108.0 g (84.9% of theory), about 97% according to ¹H-NMR.

c) Synthesis of 10-(4-methylnaphth-1-yl)-9-(phenoxy)anthracene (H1)

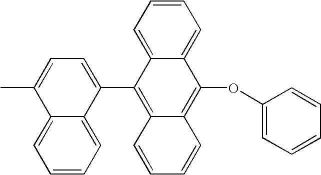

840 μl (3.5 mmol) of tri-tert-butylphosphine and then 507 mg (2.3 mmol) of palladium(II) acetate are added to a suspension of 45.0 g (113 mmol) of 9-bromo-10-(4-methylnaphth-1-yl)anthracene, 20.2 g (215 mmol) of phenol, 48.0 g (226 mmol) of tripotassium phosphate and 200 g of glass beads (diameter 0.4 mm) in 1000 ml of toluene. After the mixture has been heated at 100° C. for 12 h and cooled, 500 ml of 1 N HCl are added, and the mixture is decanted off from the glass beads. The organic phase is separated off, washed twice with 500 ml of water, dried over magnesium sulfate and filtered through silica gel with suction. The solid remaining after evaporation of the organic phase is washed with 300 ml of boiling ethanol and then recrystallised four times from DMSO (about 3 ml/g) and DMF (7 ml/g) in each case until a purity of 99.9% according to HPLC has been reached. The solid is finally sublimed under reduced pressure (p=5×10⁻⁵ mbar, T=340° C.). Yield: 17.8 g (34.2% of theory), 99.9% according to HPLC.

### Example 2

### 10-(4-Methyinaphth-1-yl)-9-(4-phenylphenoxy)anthracene (H2)

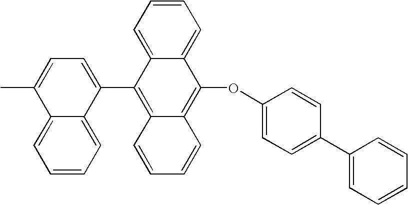

Preparation analogous to Ex. 1. Instead of phenol, 36.6 g (215 mmol) of 4-phenylphenol are used. Recrystallisation from DMSO (about 3 ml/g) and DMF (5 ml/g). Sublimation: p=5×10⁻⁵ mbar, T=355° C. Yield: 25.8 g (47.0% of theory), 99.9% according to HPLC.

### Example 3

### 10-(4-Methyinaphth-1-yl)-9-(2-phenylphenoxy)anthracene (H3)

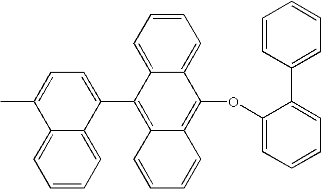

Preparation analogous to Ex. 1. Instead of phenol, 36.6 g (215 mmol) of 2-phenylphenol are used. Recrystallisation from dioxane (4 ml/g). Sublimation: p=5×10⁻⁵ mbar, T=335° C. Yield: 22.8 g (41.5% of theory), 99.9% according to HPLC.

### Example 4

### 10-(4-Methylnaphth-1-yl)-9-(9-phenanthenoxy)anthracene (H4)

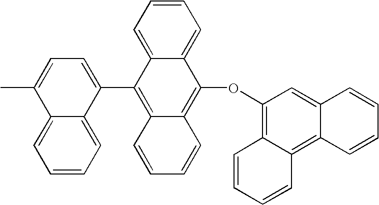

Preparation analogous to Ex. 1. Instead of phenol, 41.8 g (215 mmol) of 9-hydroxyphenanthrene are used. Recrystallisation from DMSO (5 ml/g). Sublimation: p=5×10⁻⁵ mbar, T =335° C. Yield: 29.6 g (51.3% of theory), 99.9% according to HPLC.

### Example 5

### 1-(4-Methylnaphth-1-yl)-7-(6-phenoxy)pyrene (H5)

a) Synthesis of 1-(4-methyinaphth-1-yl)pyrene

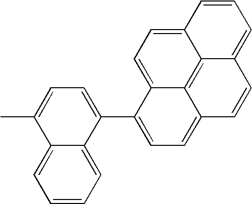

3.6 g (11.7 mmol) of tri-o-tolylphosphine and then 437 mg (1.9 mmol) of palladium(II) acetate are added to a suspension of 93.0 g (500 mmol) of 4-methylnaphthalene-1-boronic acid, 109.4 g (389 mmol) of 1-bromopyrene and 212.3 g (1 mol) of tripotassium phosphate in a mixture of 400 ml of dioxane, 600 ml of toluene and 1000 ml of water with good stirring, and the mixture is heated under reflux for 26 h. After cooling, the colourless solid is filtered off with suction, then washed by stirring with 1000 ml of hot ethanol and finally dried under reduced pressure. Yield: 119.2 g (89.5% of theory), about 97% according to ¹H-NMR.
b) Synthesis of 1-bromo-6-(4-methyinaphth-1-yl)pyrene

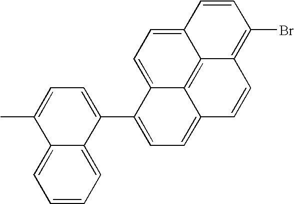

25.0 ml (220 mmol) of hydrobromic acid (aqueous, 48%) are added to a suspension of 68.5 g (200 mmol) of 1-(4-methylnaphth-1-yl)pyrene in a mixture of 500 ml of methanol and 500 ml of dibutyl ether with exclusion of light. 18.0 ml (205 mmol) of hydrogen peroxide (aqueous, 35%) are added dropwise to this mixture over the course of 4 h with good stirring. After stirring at room temperature for 12 h, the precipitate which deposits is filtered off. The resultant solid is washed with water until neutral, subsequently washed with ethanol, dried under reduced pressure and finally recrystallised twice from DMSO (about 3 ml/g). Yield: 54.5 g (64.7% of theory), about 96% according to ¹H-NMR.

c) Synthesis of 1-(4-methyinaphth-1-yl)-7-(6-phenoxy)pyrene (H5)

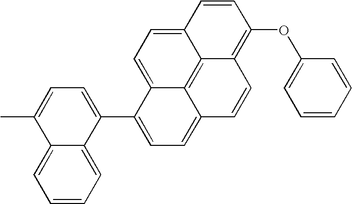

Preparation analogous to Ex. 1. Instead of 9-bromo-1 0-(4-methylnaphth-1-yl)anthracene, 47.6 g (113 mmol) of 1-bromo-6-(4-methyinaphth-1-yl)-pyrene are used. Recrystallisation from NMP (3 ml/g). Sublimation: p=5×10⁻⁵ mbar, T=380° C. Yield: 20.3 g (41.3% of theory), 99.8% according to HPLC.

### Example 6

### Production of OLEDs

OLEDs are produced by a general process as described in WO 04/05891 1 which is adapted in individual cases to the particular circumstances (for example layer-thickness variation in order to achieve optimum efficiency or colour).

In Examples 7 to 12 below, the results from various OLEDs are presented. The basic structure and the materials used (apart from the emitting layer) are identical in the examples for better comparability. Analogously to the above-mentioned general process, OLEDs having the following structure are produced:
Hole-injection layer (HIL) 20 nm PEDOT (spin-coated from water; purchased from H. C. Starck Goslar, Germany; poly(3,4-ethylenedioxy-2,5-thiophene))
Hole-transport layer (HTL) 10 nm 2,2′,7,7′-tetrakis(di-para-tolylamino)-spiro-9,9′-bifluorene (abbreviated to HTL-1)
Hole-transport layer (HTL) 30 nm NPB (N-naphthyl-N-phenyl-4,4′-diaminobiphenyl)
Emission layer (EML) see Table 1 for materials, concentration and layer thickness
Electron conductor (ETC) 20 nm AlQ₃ (purchased from SynTec, tris(quinolinato)aluminium(III))
Cathode 1 nm LiF, 150 nm Al on top.

These OLEDs are characterised by standard methods; the electroluminescence spectra, the efficiency (measured in cd/A), the power efficiency (measured in Im/W) as a function of the brightness, calculated from current/voltage/brightness characteristic lines (IUL characteristic lines), and the lifetime are determined for this purpose. The lifetime is defined as the time after which the initial brightness of 1000 cd/m² has dropped to half.

Table 1 shows the results from some OLEDs (Examples 7 to 12) which contain the host materials HO (comparative example) and H1 to H5 (examples according to the invention), with the composition of the EML including the layer thicknesses also being shown in each case. The host material HO is 9,10-bis(1-naphthyl)anthracene, and the dopant employed in all examples is D1. Both are shown below:

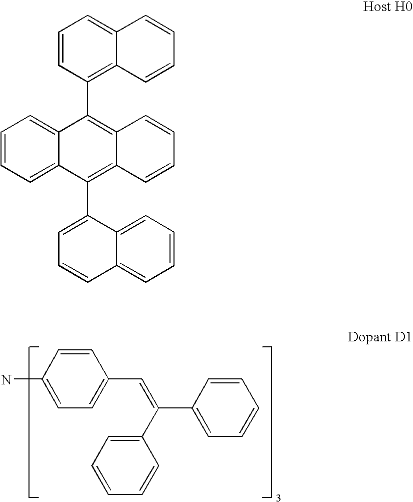

For these OLEDs, the degree of doping, i.e. the proportion of dopant in the host material, is kept constant at 5%.

As can be seen from the examples in Table 1, the tristilbenamine derivatives according to the invention exhibit blue emission with better colour coordinates and improved efficiency and significantly improved service life compared with the host material HO in accordance with the prior art.

TABLE 1

| | | Max. | Voltage | | Life- |
|---|---|---|---|---|---|
| | | efficiency | (V) at 100 | | time |
| Example | EML | (cd/A) | cd/m² | CIE | (h) |
| Example 7 | H0:D1 (5%) | 7.9 | 5.3 | x = 0.17; | 18000 |
| (comparison) | (30 nm) | | | y = 0.31 | |
| Example 8 | H1:D1 (5%) | 7.8 | 5.0 | x = 0.16; | 19000 |
| | (30 nm) | | | y = 0.30 | |
| Example 9 | H2:D1 (5%) | 8.1 | 5.1 | x = 0.16; | 19000 |
| | (30 nm) | | | y = 0.30 | |
| Example 10 | H3:D1 (5%) | 8.2 | 5.1 | x = 0.17; | 20000 |
| | (30 nm) | | | y = 0.28 | |
| Example 11 | H4:D1 (5%) | 8.2 | 4.9 | x = 0.16; | 21000 |
| | (30 nm) | | | y = 0.29 | |
| Example 12 | H5:D1 (5%) | 9.7 | 5.1 | x = 0.18; | 21500 |
| | (30 nm) | | | y = 0.32 | |

1-19. (canceled)

20. An organic electroluminescent device comprising cathode, anode and at least one organic layer comprising at least one compound of formula (1)
Ar¹—X—Ar²—Ar³ Formula (1)
wherein
Ar¹ and Ar³ is, identically or differently on each occurrence, an aromatic or heteroaromatic ring system optionally substituted by one or more R;
Ar² is, identically or differently on each occurrence, a fused aryl or heteroaryl group having at least 14 aromatic ring atoms optionally substituted by one or more R;
X is on each occurrence O, S, Se, or Te;
R is, identically or differently on each occurrence, H, F, Cl, Br, I, a straight-chain alkyl, alkoxy, or thioalkoxy chain having up to 40 C atoms and optionally substituted by R¹, or a branched or cyclic alkyl, alkoxy, or thioalkoxy chain having 3 to 40 C atoms and optionally substituted by R¹, wherein one or more non-adjacent C atoms is optionally replaced by N-R¹, O, S, O—CO—O, CO—O, Si(R¹)₂, CO, CO—N(R¹)₂, —CR¹═CR¹-, or -C≡C-, and wherein one or more H atoms is optionally replaced by F, Cl, Br, I, CN, or an aromatic or heteroaromatic ring system optionally substituted by one or more R¹, or a combination of two, three or four of these systems; and wherein two or more R optionally define a further mono- or polycyclic, aliphatic, or aromatic ring system;
R¹ is, identically or differently on each occurrence, H or an aliphatic or aromatic hydrocarbon radical having up to 20 C atoms; with the proviso that no more than one of Ar¹, Ar² and Ar³ is anthracene and with the proviso that if Ar² is anthracene, Ar¹-X is not bonded in the 2-position.

21. The organic electroluminescent device of claim 20, wherein said compound of formula (1) has a glass transition temperature T_{g} of greater than 70° C.

22. The organic electroluminescent device of claim 20, wherein Ar² contains three, four, five, or six aromatic or heteroaromatic units optionally substituted by R, which are in each case fused to one another via one or more common edges and form a common aromatic system.

23. The organic electroluminescent device of claim 20, wherein the fused aromatic or heteroaromatic units of Ar² are benzene, pyridine, pyrimidine, pyrazine, or pyridazine, each of which are optionally substituted by R.

24. The organic electroluminescent device of claim 23, wherein Ar² is selected from the group consisting of anthracene, acridine, phenanthrene, phenanthroline, pyrene, naphthacene, chrysene, pentacene, and perylene, each of which is optionally substituted by R.

25. The organic electroluminescent device of claim 20, wherein said compound of formula (1) is selected from the compounds of formulae (2) to (14), each of which is optionally substituted by R:

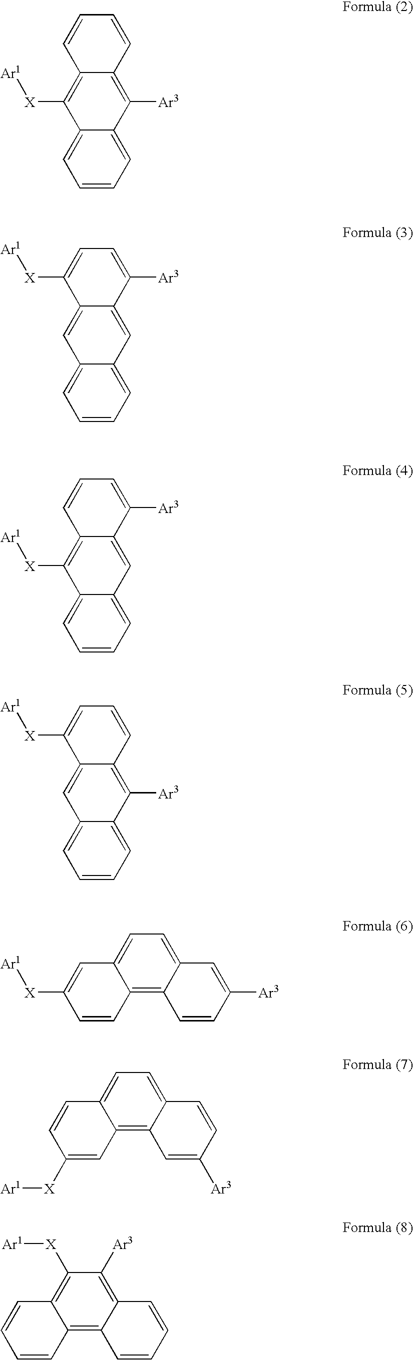

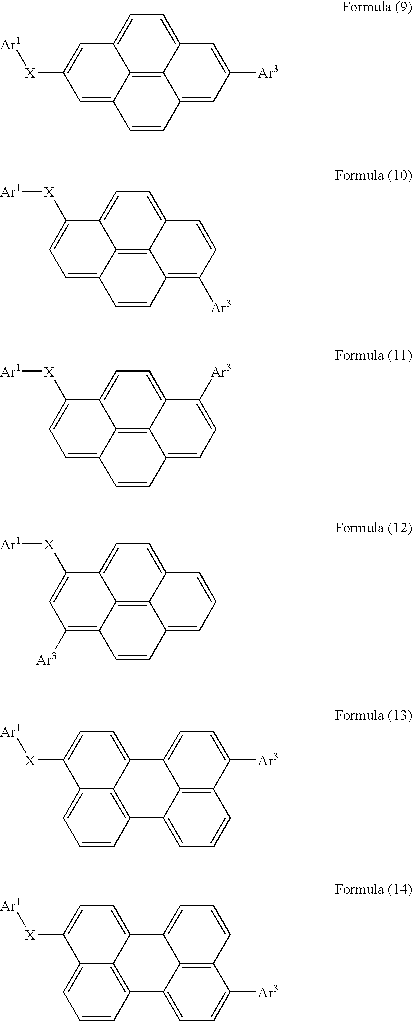

26. The organic electroluminescent device of claim 20, wherein Ar¹ and Ar³ are, identically or differently on each occurrence, simple or fused aryl or heteroaryl groups having 5 to 16 aromatic ring atoms or spirobifluorene.

27. The organic electroluminescent device of claim 20, wherein X is O or S.

28. The organic electroluminescent device of claim 20, wherein in said device comprises an emission layer and one or more additional layers selected from the group consisting of hole-injection layer, hole-transport layer, electron-transport layer, and electron-injection layer.

29. The organic electroluminescent device of claim 20, wherein said compound of formula (1) is employed in an emission layer in combination with a dopant.

30. The organic electroluminescent device of claim 29, wherein said dopant is selected from the group consisting of monostyrylamines, distyrylamines, tristyrylamines, tetrastyrylamines, and arylamines.

31. The organic electroluminescent device of claim 29, wherein said device comprises a mixture and wherein the proportion of said compound of formula (1) in said mixture is between 1 and 99.9% by weight.

32. An organic electronic device comprising one or more compounds of formula (1)
Ar¹—X—Ar²—Ar³ Formula (1)
wherein
Ar¹ and Ar³ is, identically or differently on each occurrence, an aromatic or heteroaromatic ring system optionally substituted by one or more R;
Ar² is, identically or differently on each occurrence, a fused aryl or heteroaryl group having at least 14 aromatic ring atoms optionally substituted by one or more R;
X is on each occurrence O, S, Se, or Te;
R is, identically or differently on each occurrence, H, F, Cl, Br, I, a straight-chain alkyl, alkoxy, or thioalkoxy chain having up to 40 C atoms and optionally substituted by R¹, or a branched or cyclic alkyl, alkoxy, or thioalkoxy chain having 3 to 40 C atoms and optionally substituted by R¹, wherein one or more non-adjacent C atoms is optionally replaced by N-R¹, O, S, O—CO—O, CO—O, Si(R¹)₂, CO, CO-N(R¹)₂, -CR¹═CR¹-, or -C≡C-, and wherein one or more H atoms is optionally replaced by F, Cl, Br, I, CN, or an aromatic or heteroaromatic ring system optionally substituted by one or more R¹, or a combination of two, three or four of these systems; and wherein two or more R optionally define a further mono- or polycyclic, aliphatic, or aromatic ring system;
R¹ is, identically or differently on each occurrence, H or an aliphatic or aromatic hydrocarbon radical having up to 20 C atoms;
with the proviso that no more than one of Ar¹, Ar² and Ar³ is anthracene and with the proviso that if Ar² is anthracene, Ar¹-X is not bonded in the 2-position; and
wherein said device is selected from the group consisting of organic field-effect transistors, organic thin-film transistors, organic light-emitting transistors, organic integrated circuits, organic solar cells, organic field-quench devices, and organic laser diodes

33. A compound of formula (1a)
Ar¹—X—Ar²—Ar³ Formula (1a)
wherein
Ar¹ and Ar³ is, identically or differently on each occurrence, an aromatic or heteroaromatic ring system optionally substituted by one or more R, wherein at least one of Ar¹ and Ar³ contains a fused aryl or heteroaryl group or a spirobifluorene;
Ar² is, identically or differently on each occurrence, a fused aryl or heteroaryl group having at least 14 aromatic ring atoms optionally substituted by one or more R;
X is on each occurrence O, S, Se, or Te;
R is, identically or differently on each occurrence, H, F, Cl, Br, I, a straight-chain alkyl, alkoxy, or thioalkoxy chain having up to 40 C atoms and optionally substituted by R¹, or a branched or cyclic alkyl, alkoxy, or thioalkoxy chain having 3 to 40 C atoms and optionally substituted by R¹, wherein one or more non-adjacent C atoms is optionally replaced by N-R¹, O, S, O—CO—O, CO—O, Si(R¹)₂, CO, CO—N(R¹)₂, -CR¹═CR¹-, or -C—C-, and wherein one or more H atoms is optionally replaced by F, Cl, Br, I, CN, or an aromatic or heteroaromatic ring system optionally substituted by one or more R¹, or a combination of two, three or four of these systems; and wherein two or more R optionally define a further mono- or polycyclic, aliphatic, or aromatic ring system;
R¹ is, identically or differently on each occurrence, H or an aliphatic or aromatic hydrocarbon radical having up to 20 C atoms;
with the proviso that no more than one of Ar¹, Ar², and Ar³ is anthracene and with the proviso that if Ar² is anthracene, Ar-X is not bonded in the 2-position.

34. The compounds of claim 33, wherein Ar¹ or Ar³ are selected from the group consisting of naphthalene, quinoline, isoquinoline, quinoxaline, anthracene, acridine, phenanthrene, phenanthroline, pyrene, chrysene, naphthacene, pentacene, and perylene, with the proviso that no more than one of Ar¹, Ar², and Ar³ is anthracene.

35. The compounds of claim 34, wherein Ar¹ and/or Ar³ is selected from the group consisting of naphthalene, quinoline, isoquinoline, anthracene, phenanthrene, pyrene, and perylene.

36. The compounds of claim 34, wherein Ar³ contains at least one fused aryl or heteroaryl group optionally substituted by R.

37. The compounds of claim 36, wherein Ar¹ and Ar³ contain at least one fused aryl or heteroaryl group optionally substituted by R.
